(19) United States
(12) Patent Application Publication
(10) Pub. No.: US 2026/0324961 A1
(43) Pub. Date: Oct. 22, 2026

(54) OPERATING DEVICE FOR AN ENDOSCOPE WITH A ROTARY DRUM, AN ENDOSCOPE AND A METHOD FOR OPERATING THE ENDOSCOPE

(71) Applicant: KARL STORZ SE & Co. KG, Tuttlingen (DE)

(72) Inventors: Christian GRAF, Tuttlingen (DE); Lisa PFALZGRAF, Tuttlingen (DE)

(73) Assignee: KARL STORZ SE & Co. KG, Tuttlingen (DE)

(21) Appl. No.: 19/122,033

(22) PCT Filed: Oct. 16, 2023
(86) PCT No.: PCT/EP2023/078603

§ 371 Date: Apr. 17, 2025

(30) Foreign Application Priority Data
Oct. 20, 2022 (DE) 10 2022 127 783.6

(51) Int. Cl. A61B 1/05; A61B 1/12; A61M 25/00

(52) U.S. Cl. CPC A61B 1/051; A61B 1/128; A61M 25/0075

(57) ABSTRACT

An operating device for an endoscope with a rotary drum in which an imaging system is arranged, and a hollow shaft tube extending along a longitudinal direction, wherein the operating device can be connected to the shaft tube proximally, comprising an operating housing with a displaceably received operating element, wherein the operating element is designed to interact with a control line of the rotary drum arranged on the distal side of the shaft tube in such a way that, in a first actuating portion of the operating element, a viewing panel of the imaging system is pivoted about a first transverse axis into an observation position and/or, in a second actuating portion, into a cleaning position facing the shaft tube. Furthermore, an endoscope with an operating device and an operating method for the endoscope are disclosed.

### CROSS-REFERENCE TO RELATED APPLICATIONS

This application is the U.S. national stage of PCT/EP2023/078603 filed on Oct. 16, 2023, which claims priority of German Patent Application No. DE 10 2022 127 783.6 filed on Oct. 20, 2022, the contents of which are incorporated herein.

### TECHNICAL FIELD

The present disclosure relates to an operating device for an endoscope with a rotary drum, in particular for medical applications.

### BACKGROUND

Endoscopes are used in particular for medical, preferably surgical, operations as an observation instrument, wherein the endoscope has a shaft tube with an electronic imaging system arranged on the distal side for observing an endoscope environment, in particular an operating region. To enlarge a detectable observation region, the imaging system can be arranged within a rotary drum which is pivotable about a transverse axis on the distal side of the shaft tube within a bearing fork. Preferably, a control line is arranged on the periphery of the rotary drum, which is connected to an operating device, in particular an operating handle, arranged proximally on the shaft tube, wherein a pulling movement on the control line generates a torque in order to pivot the rotary drum about the transverse axis. The control line preferably also serves to supply the imaging system electronically.

In order to cool the rear of the rotary drum, in particular the imaging system, fluid lines can be formed within the shaft tube, wherein a fluid inflow can be operated with external valve elements. Furthermore, a viewing panel of the imaging system can be pivoted from an observation position into a cleaning position facing the shaft tube, wherein the viewing panel can be exposed to a cleaning fluid and cleaned by means of the fluid lines. Preferably, a fluid suction line is also used within the shaft tube to suction out used cleaning fluid.

Exemplary embodiments of an endoscope with a rotary drum can be found in DE 10 2020 132 778 A1 or DE 10 2020 132 773 B3 of the applicant.

External valve elements for controlling fluid flow within the endoscope can lead to incorrect operation of the endoscope, since the setting position of the rotary drum cannot be coordinated with the control of the valve elements. In the observation position, this can lead to insufficient cooling of the rear of the rotary drum and damage to the electronic imaging system. Furthermore, in the cleaning position cooling fluid may be incorrectly supplied, escape from the shaft tube and contaminate a dry operating region in particular.

### SUMMARY

The present disclosure is based on the object of proposing an operating device for an endoscope which, while avoiding the problems known from the prior art, enables particularly simple, in particular one-handed, and reliable operation of an endoscope with a rotary drum.

Furthermore, the object is to indicate an endoscope with an operating device and a method for operating an endoscope.

This object is achieved with regard to the operating device with the features of independent claim 1 and with regard to the endoscope with the features of claim 12, as well as, with regard to the method for operating an endoscope, with the features of claim 13.

Advantageous embodiments are the subject matter of the subclaims.

According to the disclosure, an operating device for an endoscope is proposed, comprising a rotary drum in which an imaging system is arranged, and a hollow shaft tube extending along a longitudinal axis, wherein the operating device can be connected or is connected to the shaft tube proximally, in particular as an operating handle, comprising an operating housing with a displaceably received operating element, wherein the operating element is designed to interact with a control line of the rotary drum arranged on the distal side of the shaft tube in such a way that, in a first actuating portion of the operating element, a viewing panel of the imaging system of the rotary drum is pivoted about a first transverse axis into an observation position and/or, in a second actuating portion, into a cleaning position facing the shaft tube. The operating device comprises a valve element which interacts with the operating element and which, in the first actuating portion, selectively connects a first fluid inlet connection, in particular for a cooling fluid line, and/or, in the second actuating portion, a second fluid inlet connection, in particular for a cleaning fluid line, to at least one fluid outlet connection in order to cool the rear of the rotary drum in the observation position and/or to clean a viewing panel of the imaging system in the cleaning position in the mounted state of the operating device.

The disclosure has recognized that the interaction of the operating element with the valve element makes it possible to provide an operating device which, in the mounted state on an endoscope, can pivot a rotary drum and simultaneously control a fluid supply. A pivoting movement of the operating element can therefore be directly coupled with a fluid controller, which is why incorrect operation, in particular incorrect fluid dispensing, can be avoided. Advantageously, predefined method steps, in particular controlling the temperature of the rotary drum by means of a cooling fluid and/or a cleaning of a viewing panel of the imaging system, can be carried out particularly easily with the operating device without having to coordinate separate valve elements and pivoting mechanisms.

For cleaning the viewing panel, the cleaning fluid is preferably designed as a gaseous fluid, in particular compressed air provided externally or internally within the operating device, particularly preferably sterile air and/or carbon dioxide gas. Water can preferably be used as the cooling fluid. The cleaning fluid is preferably suitable for cleaning dirt that has entered the viewing panel of the imaging system from an operating site, in particular a front lens of an objective and/or an illumination LED of the imaging system.

A preferred imaging system comprises an illumination element, in particular an LED, and a camera element, in particular with a magnifying lens, for recording the observation region.

Particularly preferably, the operating element is designed to interact with the control line in such a way that actuating movements of the operating element and the rotary drum are directly coupled, in particular synchronized, in order to pivot the viewing panel of the imaging system into the observation position in the first actuating portion and/or into the cleaning position in the second actuating portion. In other words, the operating element interacts directly with the control line in each actuating portion in order to advantageously control the movement of the rotary drum at any time and to directly couple the fluid delivery by the valve element to the movement of the rotary drum.

In an alternative development, the operating device has a decoupling element which interacts with the operating element in order to decouple an interaction of the operating element with the rotary drum in a cleaning position in which the viewing panel is oriented along the longitudinal direction to the shaft tube, wherein in the second actuating portion the operating element is displaceable in decoupled fashion, in particular pivotable, in order to operate the valve element, in particular to open the second fluid inlet connection for the cleaning fluid. In this partially decoupled embodiment of the operating element, an outlet of the cleaning fluid can preferably be dosed and/or controlled intermittently by partially displacing the valve element, without changing the orientation of the surface to be cleaned, in particular of the viewing panel of the imaging system of the rotary drum.

In a further development, it is preferably provided that the operating element is mounted in the operating housing so as to be rotatable about a second transverse axis, wherein preferably an operating lever protrudes at least partially from the operating housing for manual operation. In particular, this enables one-handed manual operation by an operator using one finger. Manual operation has in particular the advantage that the operating device itself can be used as a disposable product and is particularly inexpensive to manufacture and mount.

Alternatively, it would also be conceivable for the operating element to be mounted within the operating housing in a translationally movable manner, wherein the operating element can act on the control line directly or by means of an additional gear arrangement when the operating device is in the mounted state. In particular, in order to set a transmission ratio between the translational movement of the operating element and the rotational movement of the rotary drum, an additional gear arrangement can be required in this embodiment.

In a preferred embodiment, the operating housing has a hollow bearing receptacle for forming the valve element, which rotatably receives a bearing portion of the operating element, wherein the first and/or the second fluid inlet connection and the at least one fluid outlet connection are formed as recesses within a peripheral wall of the bearing receptacle and wherein the bearing portion has at least one connecting channel, or forms such with an inner side of the bearing receptacle. Advantageously, by integrating the valve element into the bearing receptacle of the operating element, an operating device that is particularly easy to manufacture can be formed, wherein a separate valve element with an additional actuating mechanism can be avoided.

Preferably, the bearing element of the operating element together with the bearing receptacle forms a so-called plug valve.

In a particularly preferred development, it is provided that three recesses for a first and a second fluid inlet connection and a common fluid outlet connection are arranged in the peripheral wall of the bearing receptacle, in particular along a peripheral direction, wherein a connecting channel is formed as a segment-like cutout in the bearing element such that two recesses are connected in a fluid-conducting manner in the first and second actuating portion. The connecting channel is preferably formed together with the segment-like cutout and the inside of the bearing receptacle. Preferably, the common fluid outlet connection can simplify fluid routing within the operating device and in particular within the shaft tube. Furthermore, the manufacture of the valve element is simplified, with a smaller number of recesses and a simple connecting channel geometry, in particular without undercuts.

In other words, the connecting channel is preferably designed such that either the first or the second fluid inlet connection is connected to the fluid outlet connection. In an intermediate position, in particular a blocking portion, the segment-like cutout can be aligned with the fluid outlet connection, wherein the first and second fluid inlet connections are blocked by the bearing element itself. In this way, an emission of fluid, in particular of a cooling fluid, can be avoided when the imaging system is oriented to a wall of the shaft tube.

A common fluid outlet connection for a common fluid outlet channel within the shaft tube can have the further advantage that, in the second position, the residual cooling fluid located in the shaft tube can first be accelerated and applied to the viewing panel of the imaging system and can subsequently be applied with the cleaning fluid, in particular a gaseous fluid, in order to dry the viewing panel. In particular, drying using the cleaning fluid can remove a liquid film that is disturbing in a dry endoscope environment.

Alternatively, it would also be conceivable for the connecting channel to be formed by two bores arranged at an angle to one another within the bearing element such that, in an open position of the valve element, the first fluid inlet channel is connected to a first fluid outlet channel and/or the second fluid inlet channel is connected to a separate second fluid outlet channel. In a closed position, the connecting channel is covered on both sides by the inside of the bearing receptacle. It would also be conceivable for a plurality of connecting channels to be arranged along the second transverse axis in order to connect different fluid inlet channels with correspondingly different fluid outlet channels.

In a further preferred embodiment, the operating device has an operating element that is translationally displaceable in the operating housing for receiving the control line, wherein the rotatably mounted operating element is connected to the actuating element by means of a flexible coupling portion, in particular a film hinge, in order to transfer a rotational movement of the operating element into a translational actuating movement of the actuating element. In particular, by deforming the coupling portion, the rotational movement can be transferred into a translation. Advantageously, the flexible coupling portion can avoid a complicated joint mechanism with multiple components in order to simplify the manufacture of the operating device.

In particular, if the actuating movement of the operating element and the rotary drum are decoupled in the second actuating portion, in particular by means of the aforementioned decoupling element, and the rotary drum and/or the actuating element are in a stop position in some portions, the deformation of the coupling portion can enable a further actuating movement of the operating element. Preferably, a stop element for the translational actuating element, in particular counter to a pulling direction on the control line, can be formed in the operating housing as a decoupling element. Alternatively or additionally, a guide element for the control line can be formed within the operating housing in order to prevent a displacement of the control line in the cleaning position, in particular against a pulling direction, by increased friction.

In this context, the coupling portion, in particular of the sliding actuating element, is particularly preferably designed with a first distance and the operating lever of the operating element, in particular an operating portion for placing a thumb, is designed with a second distance from the second transverse axis, wherein a ratio, in particular a lever ratio, between the first and the second distance is between 0.17-0.5, preferably 0.25.

Advantageously, a reduction gear as a lever ratio can enable more sensitive rotation control of the rotary drum. Therefore, this embodiment of the operating device is particularly suitable for controlling rotary drums with a small diameter, wherein a small translational displacement can lead to pivoting along a large angular range.

In an alternative embodiment, the rotatably mounted operating element has an actuating element which is rotatably mounted eccentrically to the second transverse axis, wherein the operating housing forms a support portion for the control line that is loose at least along the longitudinal direction and forms a fixed support portion for the control line, wherein the eccentric actuating element is displaceable in a vertical-longitudinal plane between the loose and fixed support portions in order to, in the mounted state of the operating device, displace the control line along a vertical direction and to partially wind it up on an outer periphery of the eccentric actuating element. Preferably, in the region of the loose support portion, the operating housing has a deflecting edge on which the control line can slide during displacement along the vertical direction in order in this way to shorten the control line along the longitudinal direction and to pivot the rotary drum. Advantageously, with this embodiment the eccentric actuating element can be integrated as part of the operating element, wherein an additional mounting of the actuating element can be avoided. Furthermore, fastening the control line within the movable operating element can be avoided. Very preferably for electronic control lines, in particular a flex circuit board, an electronic contact portion can be fixedly integrated into the operating housing, in particular as part of the fixed support portion, wherein the contacting can be considerably simplified and mechanical fatigue strength can be improved.

Furthermore, in a further development the operating device can preferably have a return means, in particular a tension spring, in order to interact with a return line, in particular a filament, of the rotary drum, preferably in such a way that in an initial position of the return means the rotary drum is in the cleaning position. Very preferably, the return means are used in conjunction with the aforementioned eccentric actuating element in order to pre-tension the control line for displacement along the vertical direction. This allows the control line to be wound more reliably onto the eccentric actuating element.

The return means can alternatively or additionally be designed as a spiral spring. Furthermore, in an alternative embodiment, it is conceivable for the return line to form the return means itself as an elastic filament.

Particularly preferably, the return means are designed to pre-tension the electronic control line in order to avoid compressive stress on the control line, in particular to avoid kinking and damage to the electronic control line.

In a further preferred embodiment, the operating device has a pump element, in particular a syringe element, for storing and conveying a fluid, in particular a cleaning fluid, wherein the pump element is connected to the second fluid inlet connection of the valve element and wherein the operating element interacts with the pump element in such a way that, before reaching the second actuating portion, in particular in a cleaning actuating movement, the operating element compresses the fluid in the pump element and, in the second actuating portion the valve element opens the pump element for cleaning the rotary drum.

Advantageously, with this embodiment a fluid can be conveyed by means of the operating device without requiring an external connection, in particular a compressed air line. Furthermore, particularly intuitive operation can be enabled, wherein no additional actuation of a separate, in particular external, operating element is necessary.

Most preferably, the pump element is designed to suction in ambient air, in particular by means of a check valve, which air can be compressed by the pump element in order to convey the cleaning fluid in the direction of the rotary drum when the valve element is open.

Preferably, the operating element acts partially on the pump element in the first actuating portion in order to increase a pressure within the pump element.

In this context, it is particularly preferred that the pump element is designed as a cylinder with a piston, fastened in the operating housing, wherein a return means, in particular a compression spring, is arranged within the cylinder in order to move the piston into an initial position of the piston after compression and in the process to suction in the fluid, in particular by means of a check valve. Advantageously, the integration of the pump element within the operating housing enables operation of the endoscope at least with a reduced number of external fluid connections and pressure sources that would have to be controlled separately. Very particularly preferably, the pump element is designed as a syringe, in particular a syringe known from the medical field, which can be manufactured and mounted at particularly low cost.

The disclosure also relates to an endoscope with an operating device, in particular an aforementioned operating device, and with a rotary drum in which an imaging system is arranged, and a hollow shaft tube extending along a longitudinal axis, wherein the operating device is connected to the shaft tube proximally, in particular as an operating handle, and comprises an operating housing with a displaceably received operating element, wherein the operating element interacts with a control line of the rotary drum arranged on the distal side of the shaft tube in such a way that in a first actuating portion of the operating element the imaging system of the rotary drum is pivoted about a first transverse axis into an observation position and in a second actuating portion into a cleaning position facing the shaft tube. The operating device comprises a valve element which interacts with the operating element and which selectively opens, in the first actuating portion, a cooling fluid line, and/or, in the second actuating portion, a cleaning fluid line, of the endoscope in order to cool the rear of the rotary drum in the observation position and/or to clean a viewing panel of the imaging system in the cleaning position.

Furthermore, the disclosure also relates to a method for operating an endoscope with an operating device, in particular an aforementioned operating device, wherein the endoscope is provided with a rotary drum in which an imaging system is arranged and a hollow shaft tube extending along a longitudinal direction, wherein the operating device is connected proximally to the shaft tube, in particular as an operating handle, which forms an operating housing with an operating element and a valve element, wherein the operating element pivots the rotary drum by means of a control line. In a preferred first method step, the operating element is moved into a first actuating portion, in particular along an observation actuating direction, in order to pivot the imaging system of the rotary drum into an observation position, wherein the valve element opens a first fluid inlet connection of a cooling fluid line in order to cool the rear of the rotary drum. In a preferred second additional or alternative method step, in a cleaning position of the rotary drum in which the imaging system is pivoted in the direction of the shaft tube, the operating element is displaced in a second actuating portion and opens a second fluid inlet connection of the valve element in order to clean a viewing panel of the imaging system.

Particularly preferably, in the second method step the operating element is moved in the second actuating portion coupled to the rotary drum, in particular along a cleaning actuating direction, in order to pivot the imaging system of the rotary drum into the cleaning position facing the shaft tube.

In a preferred development of the method, during an actuating movement along a cleaning actuating direction the operating element acts on a pump element within the operating housing, which pump element is connected to the second fluid inlet connection of the valve element, wherein the operating element, during the actuating movement, before reaching the second actuating portion in the direction of the cleaning position, compresses a cleaning fluid stored in the pump element, in particular previously suctioned-in air, wherein in the second actuating portion the valve element opens the pump element in order to conduct the previously compressed cleaning fluid to the rotary drum and to clean the viewing panel of the imaging system.

Further advantages and details of the disclosure can be seen from the following description of preferred embodiments of the disclosure and from the drawings, which are merely schematic.

### BRIEF DESCRIPTION OF DRAWINGS

FIG. 1 is a side view of an endoscope with a shaft tube, a distal-side rotary drum and a proximal operating device,

FIG. 2 is a side view of the rotary drum according to FIG. 1 in a cleaning position,

FIG. 3a is a cross-sectional view of the rotary drum according to FIG. 2 in an observation position,

FIG. 3b is a side view of the operating device according to FIG. 1 with first and second actuating portions,

FIG. 3c is a side view of the operating device according to FIG. 3b with detail views of a valve element,

FIG. 4a,b are a side view and a front view of the operating device according to FIG. 1 with a translationally displaceable actuating element for a control line,

FIG. 5a to FIG. 5d are cross-sectional views of the rotary drum and the operating device according to FIG. 1 and FIG. 2 in an observation position and a cleaning position,

FIG. 6a,b are schematic side views of an operating element with an eccentrically arranged actuating element,

FIG. 7 is a side view of an operating device with the eccentric actuating element according to FIG. 6a,b,

FIG. 8a to FIG. 8d are side views of an operating device according to FIG. 1 with an operating element acting on a pump element,

FIG. 9a to FIG. 9f are cross-sectional views of the rotary drum and the operating device according to FIG. 7 in an actuating movement from a first to a second actuating portion.

Identical elements or elements with the same function are marked with the same reference signs in the figures.

### DETAILED DESCRIPTION OF PREFERRED EMBODIMENTS

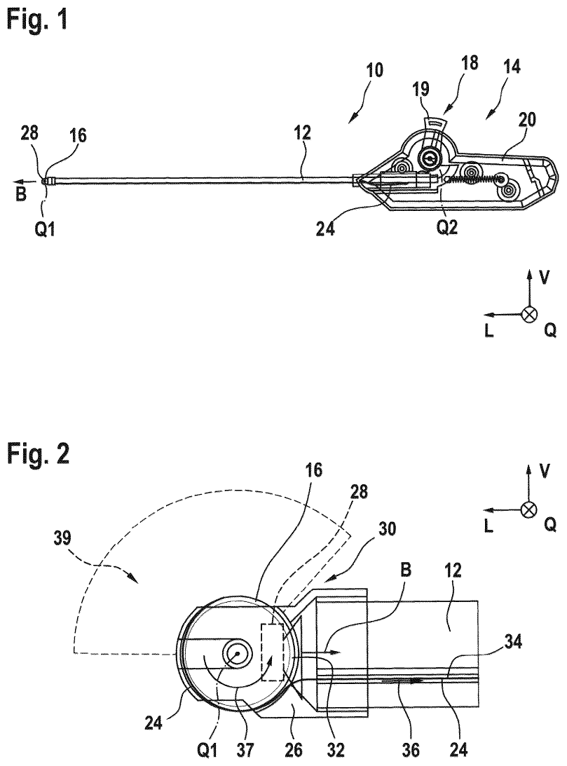

FIG. 1 shows a side view of an endoscope 10 with a hollow shaft tube 12 extending along a longitudinal direction L, wherein an operating device 14 is shown in the mounted state proximally of the shaft tube 12 and wherein a rotary drum 16 is arranged on the distal side of the shaft tube 12. Preferably, the operating device 14 serves as an operating handle to pivot the rotary drum 16 about a first rotation axis Q1 and to adjust an observation direction B of an imaging system 28 arranged within the rotary drum 16. For this purpose, the operating device 14 preferably has an operating element 18 which is mounted in an operating housing 20 so as to be rotatable about a second transverse axis Q2 and which interacts with a control line 24 to pivot the rotary drum 16. For manual operation, in particular one-handed operation, the operating element 18 preferably has an operating lever 19 that protrudes at least partially from the operating housing 20.

In FIG. 2, the rotary drum 16 is shown in detail, wherein a bearing fork 26 connects the rotary drum 16 to the shaft tube 12 so as to be pivotable about the first transverse axis Q1. The pivotable imaging system 28 is arranged in the rotary drum 16, wherein in a cleaning position 30 the viewing direction B is directed towards the shaft tube 12. Here a cleaning fluid conducted through the interior of the shaft tube 12 can act on and clean a viewing panel 32 of the imaging system 28 of the rotary drum 16. Furthermore, the control line 24 is shown here, which is preferably fastened on the periphery to the rotary drum 16 and is preferably guided in a working channel 34 of the hollow shaft tube 12. Here a pulling movement 36 leads to the application of a torque to the rotary drum 16 and thus to a rotational movement, in particular along an observation actuating direction 37, in order to pivot the viewing direction B towards an observation position 39 facing away from the shaft tube 12, wherein the observation position 39 is schematically shown as a dashed observation actuating portion or observation angle portion about the first transverse axis Q1. The rotary drum 16 can be pivoted until a pure tensile force acts on the rotary drum 16 due to unwinding of the control line 24.

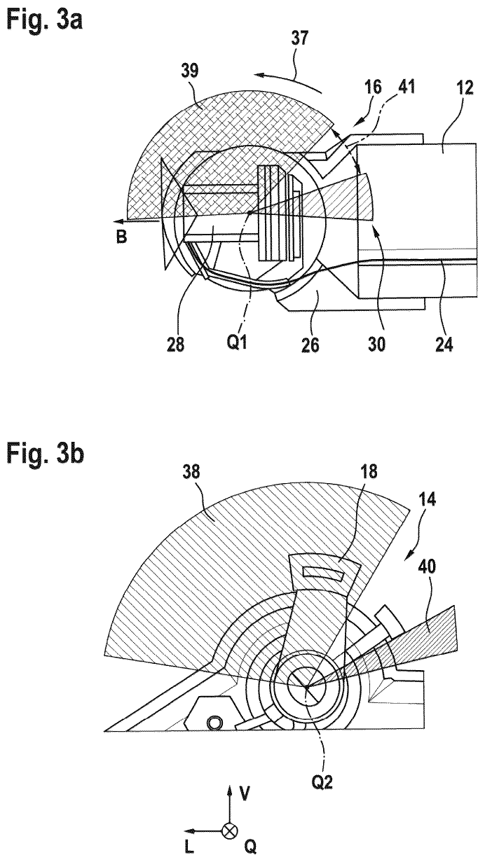

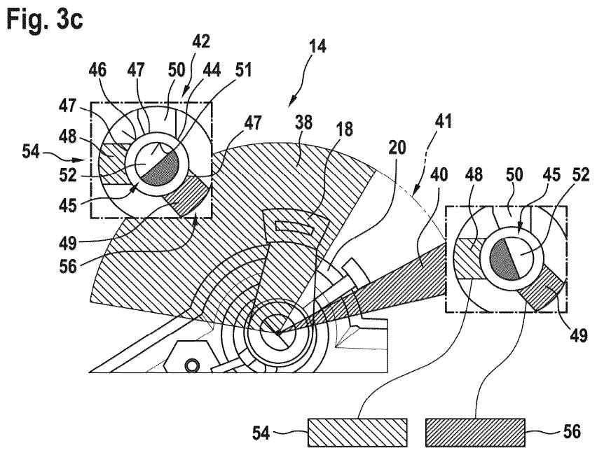

In FIG. 3a and FIG. 3b, the previously shown rotary drum 16 is shown in detail together with the operating element 18 of the operating device 14, wherein coupled actuating portions, in particular actuating angle portions with respect to the first and second transverse axes Q1, Q2, of the operating element 18 and the rotary drum 16 are shown. In a first actuating portion 38 of the operating element 18, the rotary drum 16 can be pivoted into the observation position 39 and, in a second actuating portion 40 of the operating element 18, the viewing direction B can be pivoted about the first transverse axis Q1 into the cleaning position 30 facing the shaft tube 12. Between the first and second actuating portions 38, 40, the viewing direction B is covered by the bearing fork 26 in a blocking portion 41.

Furthermore, it can be seen from FIG. 3a that the previously described rotational movement, in particular along the observation actuating direction 37, of the rotary drum 16 is preferably limited to a half-side rotation portion, wherein the endoscope 10 itself can be rotated about the longitudinal axis L to expand the observation region.

FIG. 3c shows a detailed view of the operating device 14 with operating element 18 and detail views of a valve element 42. The valve element 42 is preferably formed within the operating housing 20 as a hollow bearing receptacle 44 into which the operating element 18 rotatably engages with a bearing portion 45. In a peripheral wall 46 of the bearing receptacle 44, a plurality of, in particular three, recesses 47 are arranged to preferably accommodate a first and a second fluid inlet connection 48, 49 and a fluid outlet connection 50. Within the bearing receptacle 44, the bearing element 45 forms a connecting channel 52, in particular with an inner side 51 of the bearing receptacle 44. In the first actuating portion 38, the connecting channel 52 connects the first fluid inlet connection 48, in particular a cooling fluid line 54, and in the second actuating portion 40 connects the second fluid inlet connection 49, in particular a cleaning fluid line 56, to the fluid outlet channel 50 in order to cool the rear of the rotary drum 16 in the observation position 39 and to apply the cleaning fluid to the viewing panel 32 of the imaging system 28 in the cleaning position 30. In a preferred embodiment, the connecting channel 52 is formed as a segment-like cutout in the bearing element 45, in particular as a semicircular cutout, wherein in each case two of the three recesses 47 can be connected in a fluid-conducting manner. In the blocking portion 41, the connecting channel 52 is aligned in the direction of the fluid outlet connection 50 and thus blocks the first and second fluid inlet connections 48, 49.

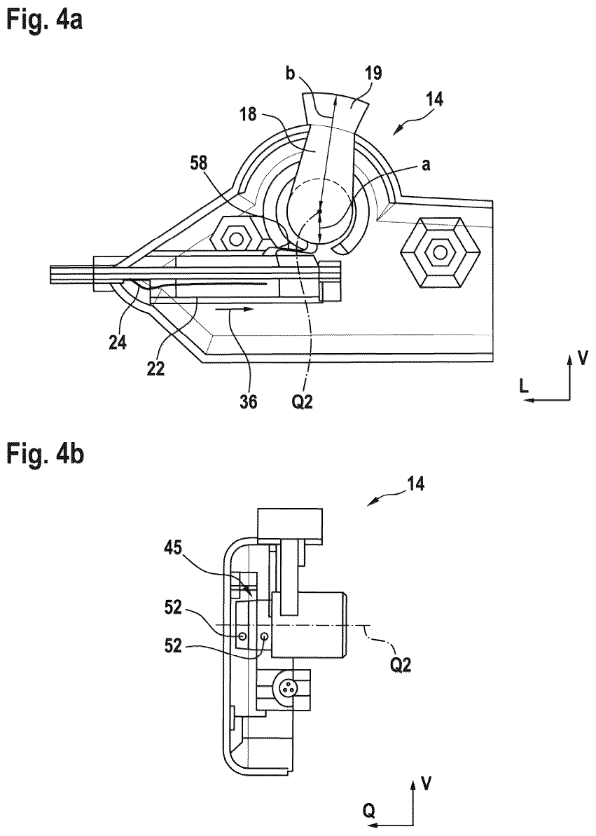

In FIG. 4a and FIG. 4b, the operating device 14 according to FIG. 1 is shown in detail in a first embodiment, wherein the operating element 18 is mounted rotatably about the second rotation axis Q2. The operating element 18 has an actuating element 22 which is mounted in the operating housing 20 in a translationally displaceable manner and in which the control line 24 of the rotary drum 16 can be fixed. In order to transfer a rotational movement of the operating element 18 into a translational actuating movement of the actuating element 22, the operating element 18 additionally has a flexible coupling portion 58, in particular a film hinge. In particular, a deformability of the coupling portion 58 enables the transfer of a torque into a pulling movement 36 for displacing the control line 24 along the longitudinal direction L. Further advantageously, a reduction gear between the operating element 18 and a movement of the control line 24 can be set by means of the coupling portion 58. Thus, the coupling portion 48 can be arranged at a first distance a and the operating lever 19 of the operating element 18, in particular an operating portion of the operating lever for placing a thumb, at a second distance b from the second transverse axis Q2, wherein a ratio between the first and the second distance a, b is preferably between 0.17 and 0.5, particularly preferably 0.25. This reduction gear can enable more sensitive operation of the rotary drum 16.

FIG. 4b shows the operating device 14 in a vertical-transverse plane V-Q, illustrating that the bearing element 45 can be formed along the transverse axis Q2. It is conceivable here that, in an additional or alternative embodiment, a plurality of connecting channels 52 can also be arranged along the transverse axis Q2 within the bearing element 45 in order to be able to connect a plurality of fluid inlet connections 48, 49 (not shown here) to a plurality of fluid outlet connections 50.

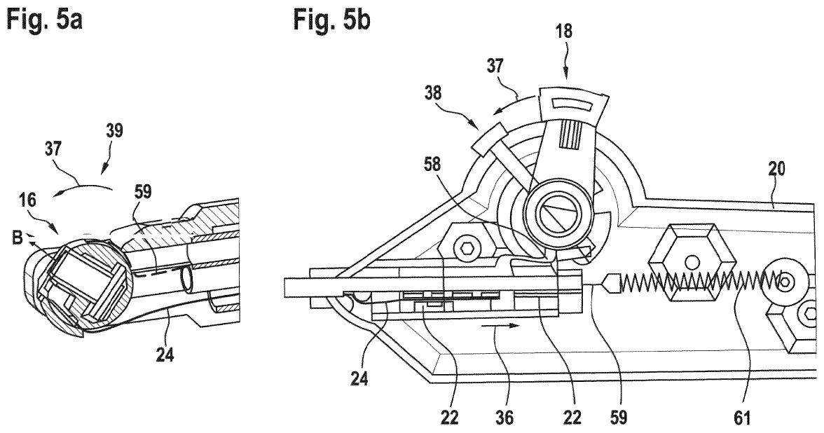

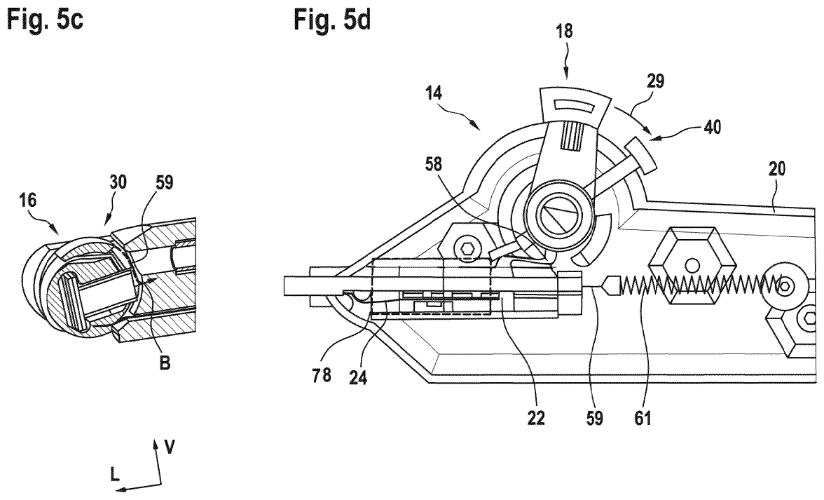

FIG. 5a to FIG. 5d show the previously described operating device 14 with actuating element 22 in the first and second actuating portions 38, 40 of the operating element 18.

FIG. 5a and FIG. 5b show the operating element 18 in the first actuating portion 38 and an observation actuating movement along the observation actuating direction 37, wherein the actuating element 22 moves the control line 24 and thus the rotary drum 16 with the viewing direction B into the observation position 39 along the pulling direction 36. In addition to an initial position, the operating element 18 is schematically shown in FIG. 5b in a position pivoted along the observation actuating direction 37.

FIG. 5c and FIG. 5d show the operating element 18 in a cleaning actuating movement along a cleaning actuating direction 29 towards the second actuating portion 40 in order to move the rotary drum 16 into the cleaning position 30. In addition to the control line 24, a return line 59 is preferably arranged, in particular fastened, on the periphery of the rotary drum 16 in order to guide the rotary drum 16 into the cleaning position 30. For this purpose, a return means 61, in particular in the form of a tension spring, is preferably fastened to the return line 59 within the operating housing 20.

In FIG. 5d, an embodiment of the operating device 14 can also be seen, wherein the rotary drum 16 in the cleaning position 30 is aligned in particular along the longitudinal direction L, and a further actuating movement of the operating element 18 along the cleaning actuating direction 29 within the second actuating portion 40 is decoupled from the actuating movement of the rotary drum 16. For decoupling, a decoupling element 78, in particular a stop element, can be arranged in the operating housing 20 in order to limit the translational actuating movement of the actuating element 22 counter to the pulling direction 36. In particular, the previously described flexible coupling portion 58 a further decoupled actuating movement of the operating element 18 relative to the actuating element 22 resting on the decoupling element 78 can be enabled by deformation. Alternatively or additionally, it is conceivable that a guide geometry for the control line 24 is formed within the operating housing 20 in such a way that, when there is an actuating movement counter to the pulling direction 36, an increased friction on the control line 24 can prevent a longitudinal displacement of the control line 24 within the shaft tube 12. In addition to an initial position, the operating element 18 is schematically shown in FIG. 5d in a position pivoted along the cleaning actuating direction 29.

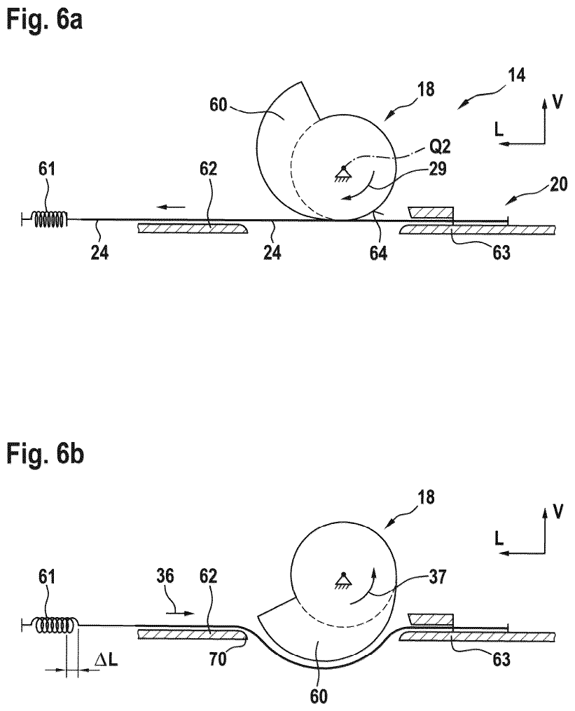

In FIG. 6a and FIG. 6b, a second embodiment of the operating element 18 is schematically shown with an actuating element 60 which is rotatably mounted eccentrically to the second transverse axis Q2, wherein the eccentric arrangement of the actuating element 60 enables an actuating movement of the operating element 18 along the vertical direction V. In this second embodiment, the operating housing 20 preferably has a loose and a fixed support portion 62, 63 for the control line 24, wherein the eccentrically mounted actuating element 60 is movable in the vertical-longitudinal plane V-L between the loose and fixed support portions 62, 63 in order to displace the control line 24 along the vertical direction V and to partially wind it up on an outer periphery 64 of the eccentric actuating element 60. During the actuating movement, the control line 24 can slide along the loose support portion 62, in particular a deflecting edge 70 within the operating housing 20, and thus enables the pulling movement 36 on the control line 24, in particular by a change in length ΔL, in order to perform the observation actuating movement along the observation actuating direction 37 of the rotary drum 16 according to FIG. 2.

FIG. 6a also schematically shows the return means 61, which enables a return of the control line 24, in particular with a return line 59 (not shown). Preferably, the eccentric actuating element 60 can be positioned along the cleaning actuating direction 29 from the first to the second actuating portion 38, 40 and wherein the return line 59 rotates the rotary drum 16. Very particularly preferably, the control line 24 remains tensioned by the return means 61 and thus enables uninterrupted guidance on the outer periphery 64 of the eccentric actuating element 60.

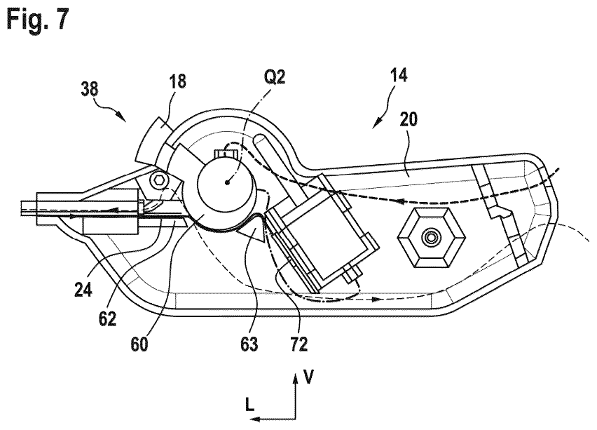

An exemplary embodiment of the eccentric actuating element 60 is shown in FIG. 7, wherein loose and fixed support portions 62, 63 are formed within the operating housing 20 and wherein in the first actuating portion 38 of the operating element 18 shown here, the control line 24 is displaced vertically between the loose and fixed support portions 62, 63 by pivoting about the second transverse axis Q2.

Furthermore, FIG. 7 shows a preferred control line 24 with an electronic contact portion 72 for the electronic supply of the imaging system 28, which can particularly preferably be fixed firmly in the operating housing 20, in particular adjacent to the fixed support portion 63, and/or can be contacted electronically. The electronic contact portion 72 is advantageously mounted immovably and independently of the actuating movement of the operating element 18 and is protected from varying and potentially damaging mechanical stress. In other words, the control line 24 is advantageously not fixedly connected to the operating element 18, but interacts with the operating element 18 by being wound up, wherein the control line 24 itself is fixedly connected on one side to the operating housing 20, in particular to the fixed support portion 63.

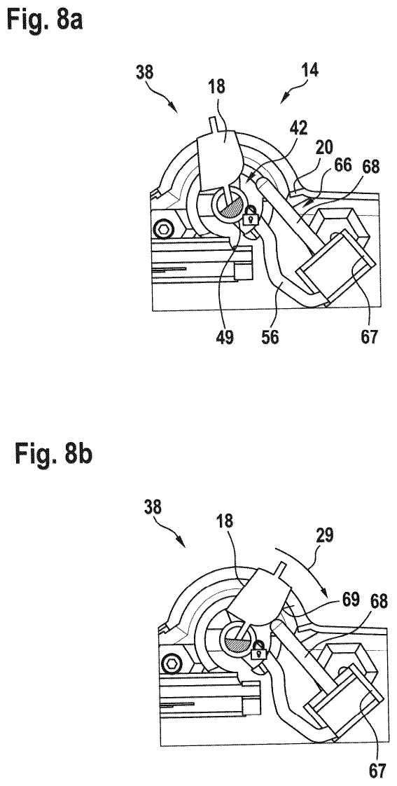

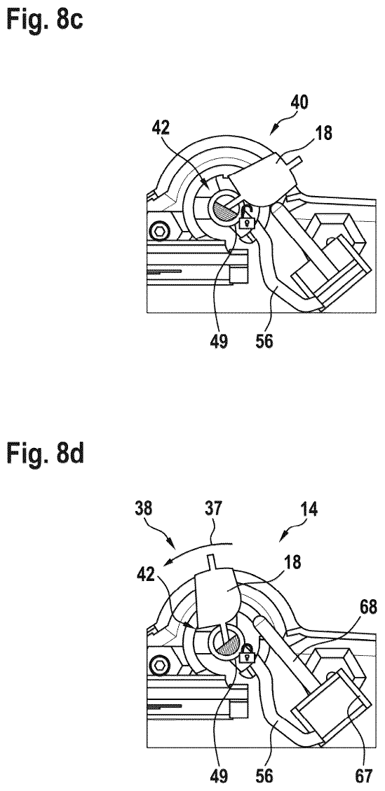

FIG. 8a to FIG. 8d show a further particularly preferred embodiment of the operating device 14, wherein the valve element 42 interacts with a pump element 66. The pump element 66 is preferably arranged as a cylinder 67 with a displaceable piston 68 within the operating housing 20. On a lower side of the cylinder 67, the pump element 66 is connected to the second fluid inlet connection 49 of the valve element 42 by a cleaning fluid line 56.

As shown in FIG. 8b, the operating element 18 preferably acts on the displaceable piston 68 along the cleaning actuating direction 29 starting from the first actuating portion 38; in particular the operating element 18 forms a sliding surface 69 in order to transfer the rotational movement of the operating element 18 into a translational actuating movement of the piston 68, in particular with a piston rod. Before reaching the second actuating portion 40, the cylinder 67 is closed and a fluid stored therein can be compressed.

In FIG. 8c, the operating element 18 is located in the second actuating portion 40 and thus the cleaning position 30 of the rotary drum 16, wherein the valve element 42 is open and conducts the fluid compressed in the cylinder 67 for cleaning the viewing panel 32 in the direction of the rotary drum 16.

In FIG. 8d, the operating element 18 is moved back along the observation actuating direction 37 into the first actuating portion 38. A compression spring (not shown here) can be arranged within the cylinder 67 in order to move the piston 68 into an initial position according to FIG. 8b and to suction in a cleaning fluid.

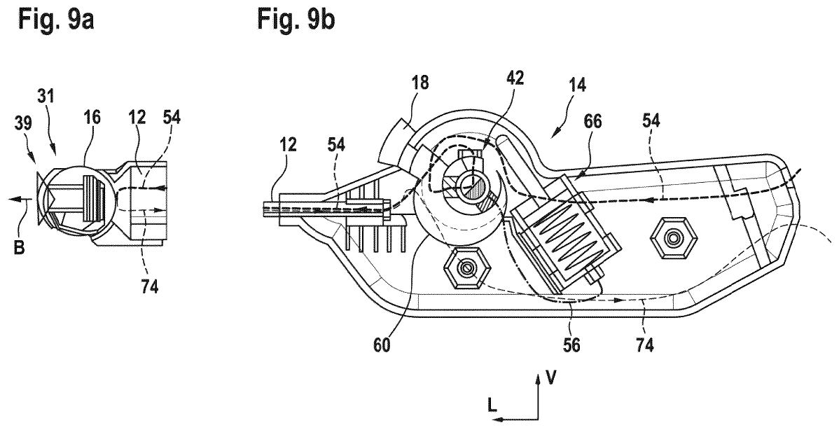

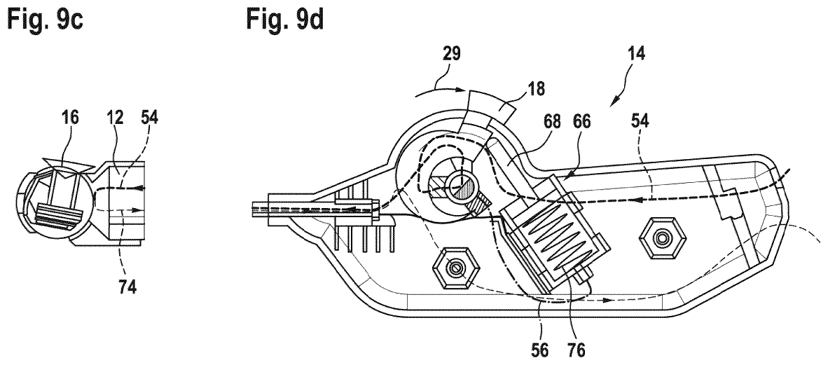

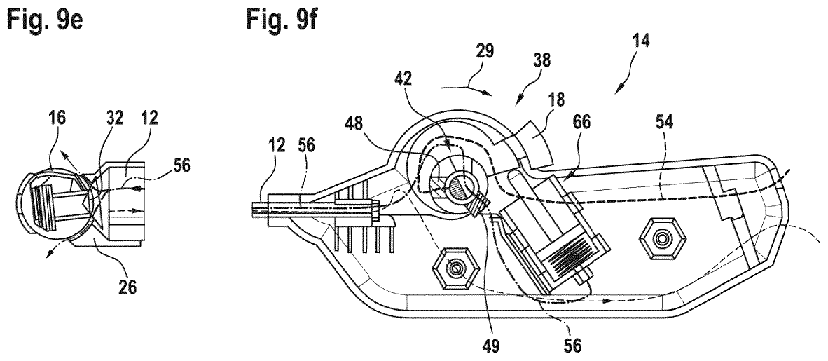

In FIG. 9a to FIG. 9f, the rotary drum 16 and the operating device 14 with the operating element 18 and the eccentric actuating element 60 according to FIG. 7 are shown during the cleaning actuating movement along the cleaning actuating direction 29. Here the operating element 18 acts on the pump element 66 analogously to FIG. 8a to FIG. 8c.

In FIG. 9a and FIG. 9b, the rotary drum 16 is in the observation position 39, wherein the cooling fluid line 54 is opened by the valve element 42 and is guided through the shaft tube 12 for cooling the rear of the rotary drum 16. Furthermore, there is preferably also a suction line 74 within the shaft tube 12, which drains the cooling fluid and thus prevents it from escaping from the shaft tube 12.

In FIG. 9c and FIG. 9d, the operating element 18 is moved in the cleaning actuating direction 29 and in the process acts on the pump element 66 in order to compress the cleaning fluid stored therein and to displace the piston 68 against the compression spring 76.

In FIG. 9e and FIG. 9f, the operating element 18 is located within the second actuating portion 38, wherein the valve element 42 closes the first fluid inlet connection 48 for the cooling fluid line 54, in particular a cooling water line, and opens the second fluid inlet connection 49 for the cleaning fluid line 56 in order to convey the cleaning fluid compressed in the pump element 66 through the shaft tube 12 to the rotary drum 16 and to clean the viewing panel 32. It is conceivable for the cleaning fluid to exit from the shaft tube 12 or the bearing fork 26.

1. An operating device for an endoscope with a rotary drum in which an imaging system is arranged, and a hollow shaft tube extending along a longitudinal direction, wherein the operating device can be connected or is connected to the shaft tube proximally, in particular as an operating handle, comprising an operating housing with a displaceably received operating element, wherein the operating element is designed to interact with a control line of the rotary drum arranged on the distal side of the shaft tube in such a way that, in a first actuating portion of the operating element, a viewing panel of the imaging system is pivoted about a first transverse axis into an observation position and/or, in a second actuating portion, into a cleaning position facing the shaft tube, wherein the operating device comprises:
a valve element which interacts with the operating element and which, in the first actuating portion, selectively connects a first fluid inlet connection, in particular for a cooling fluid line and/or, in the second actuating portion, a second fluid inlet connection, in particular for a cleaning fluid line, to at least one fluid outlet connection in order to cool the rear of the rotary drum in the observation position and/or to clean a viewing panel of the imaging system in the cleaning position, in the mounted state of the operating device.

2. The operating device according to claim 1, wherein the operating element is designed to interact with the control line in such a way that actuating movements of the operating element and the rotary drum are directly coupled in order to pivot the viewing panel of the imaging system into the observation position in the first actuating portion and/or into the cleaning position in the second actuating portion.

3. The operating device according to claim 1, wherein the operating element is mounted in the operating housing so as to be rotatable about a second transverse axis, wherein preferably an operating lever protrudes at least partially from the operating housing for manual operation.

4. The operating device according to claim 3, wherein the operating housing has a hollow bearing receptacle for forming the valve element, which receptacle rotatably receives a bearing element of the operating element, wherein the first and/or the second fluid inlet connection and the at least one fluid outlet connection are formed as recesses within a peripheral wall of the bearing receptacle and wherein the bearing element has at least one rotatable connecting channel or forms such with an inner side of the bearing receptacle.

5. The operating device according to claim 4, wherein three recesses for a first and a second fluid inlet connection and a common fluid outlet connection are arranged in the peripheral wall of the bearing receptacle, wherein a connecting channel is formed as a segment-like cutout in the bearing element such that two recesses are connected in a fluid-conducting manner in the first and second actuating portion.

6. The operating device according to claim 3, characterized in that
the operating device has an actuating element which is translationally displaceable in the operating housing for receiving the control line, wherein the rotatably mounted operating element is connected to the actuating element by means of a flexible coupling portion, in particular a film hinge, in order to transfer a rotational movement of the operating element into a translational actuating movement of the actuating element.

7. The operating device according to claim 6, wherein the coupling portion is formed with a first distance and an operating lever of the operating element is formed with a second distance from the second transverse axis, wherein a ratio between the first and the second distance is between 0.17 and 0.5, preferably 0.25.

8. The operating device according to claim 3, wherein the rotatably mounted operating element has an actuating element which is rotatably mounted eccentrically to the second transverse axis, wherein the operating housing forms a support portion for the control line that is loose at least along the longitudinal direction and forms a fixed support portion for the control line, wherein the eccentric actuating element is displaceable in a vertical-longitudinal plane between the loose and fixed support portions in order to, in the mounted state of the operating device, displace the control line along a vertical direction and to partially wind it up on an outer periphery of the eccentric actuating element.

9. The operating device according to claim 1, wherein the operating device has a return means, in particular a tension spring, in order to interact with a return line of the rotary drum, preferably in such a way that in an initial position of the return means the rotary drum is in the cleaning position.

10. The operating device according to claim 1, wherein the operating device has a pump element for storing and conveying a fluid, in particular a cleaning fluid, wherein the pump element is connected to the second fluid inlet connection of the valve element and wherein the operating element interacts with the pump element in such a way that, before reaching the second actuating portion, the operating element compresses the fluid in the pump element and, in the second actuating portion, the valve element opens the pump element for cleaning the rotary drum.

11. The operating device according to claim 10, wherein the pump element is designed as a cylinder with a piston fastened in the operating housing, wherein a compression spring is arranged within the cylinder in order to move the piston into an initial position after a compression and in the process to suction in the fluid.

12. An endoscope with an operating device, in particular according to one of claims 1 to 11, a rotary drum in which an imaging system is arranged, and a hollow shaft tube extending along a longitudinal axis, wherein the operating device is connected to the shaft tube proximally, in particular as an operating handle, wherein the operating device comprises an operating housing with a displaceably received operating element, wherein the operating element interacts with a control line of the rotary drum arranged on the distal side of the shaft tube in such a way that in a first actuating portion of the operating element, the imaging system of the rotary drum is pivoted about a first transverse axis into an observation position, and in a second actuating portion into a cleaning position facing the shaft tube, wherein the operating device comprises:
a valve element which interacts with the operating element in such a way that, in the first actuating portion, the valve element selectively opens a cooling fluid line and/or, in the second actuating portion, a cleaning fluid line of the endoscope in order to cool the rear of the rotary drum in the observation position and/or to clean a viewing panel of the imaging system in the cleaning position.

13. A method for operating an endoscope with an operating device, in particular according to claims 1 to 11, wherein the endoscope is provided with a rotary drum in which an imaging system is arranged, and a hollow shaft tube extending along a longitudinal direction, wherein the operating device is connected proximally to the shaft tube, in particular as an operating handle, which forms an operating housing with an operating element and a valve element, wherein the operating element pivots the rotary drum by means of a control line, wherein the operating element is moved in a first actuating portion in order to pivot the imaging system of the rotary drum into an observation position, wherein the valve element opens a first fluid inlet connection of a cooling fluid line in order to cool the rear of the rotary drum, and/or wherein in a cleaning position of the rotary drum, in which the imaging system is pivoted in the direction of the shaft tube, the operating element is displaced in a second actuating portion, in particular along a cleaning actuating direction, and opens a second fluid inlet connection of the valve element in order to clean a viewing panel of the imaging system.

14. The method according to claim 13, wherein during an actuating movement along a cleaning actuating direction the operating element acts on a pump element within the operating housing, which pump element is connected to the second fluid inlet connection of the valve element, wherein the operating element, during the actuating movement before reaching the second actuating portion, compresses a cleaning fluid stored in the pump element, in particular previously suctioned-in air, wherein in the second actuating portion the valve element opens the pump element in order to conduct the previously compressed cleaning fluid to the rotary drum and to clean the viewing panel of the imaging system.
